(19) United States
(12) Patent Application Publication
(10) Pub. No.: US 2023/0416753 A1
(43) Pub. Date: Dec. 28, 2023

(54) SIRNA FOR TREATING HEPATIC FIBROSIS AND DELIVERY PREPARATION THEREOF

(71) Applicant: YOUJIA (HANGZHOU) BIOMEDICAL TECHNOLOGY CO., LTD, Hangzhou (CN)

(72) Inventors: Wenhao CUI, Hangzhou (CN); Chengjiang ZHAO, Hangzhou (CN); Meng WANG, Hangzhou (CN); Zhi ZHENG, Hangzhou (CN); Huandong TIAN, Hangzhou (CN)

(73) Assignee: YOUJIA (HANGZHOU) BIOMEDICAL TECHNOLOGY CO., LTD, Hangzhou (CN)

(21) Appl. No.: 18/037,598

(22) PCT Filed: Nov. 19, 2021
(86) PCT No.: PCT/CN2021/131951

§ 371 Date: May 18, 2023

(30) Foreign Application Priority Data
Nov. 19, 2020 (CN) 202011302953.5

(51) Int. Cl. C12N 15/113; A61K 47/34; A61K 9/08; A61K 31/713; A61P 1/16; A61P 11/00

(52) U.S. Cl. CPC C12N 15/1137; A61K 47/34; A61P 11/00; A61K 31/713; A61P 1/16; A61K 9/08

(57) ABSTRACT

Provided is a siRNA targeting a NOX1 protein for inhibiting the production of reactive oxygen species (ROS) to treat hepatic fibrosis. In addition, the siRNA has an improved effect on the treatment of hepatic fibrosis and non-alcoholic fatty liver.

### CROSS-REFERENCE TO RELATED APPLICATIONS

This is a U.S. National Phase application under 35 U.S.C. § 371 of International Application No. PCT/CN2021/131951, filed on Nov. 19, 2021, which claims the priority of Chinese Patent Application No. 202011302953.5, filed on Nov. 19, 2020, and titled with “SIRNA FOR TREATING HEPATIC FIBROSIS AND DELIVERY PREPARATION THEREOF”, and the disclosure of which is hereby incorporated by reference.

### INCORPORATION OF SEQUENCE LISTING

The sequence listing that is contained in the file named “UNIT-P0102US-Sequence_Listing”, which is 4 KB (as measured in Microsoft Windows®) and created on June 19, 2023, is filed herewith by electronic submission, and is incorporated by reference herein.

### FIELD

The present invention relates to the field of pharmaceutical preparations, in particular to an siRNA for treating hepatic fibrosis and a delivery preparation thereof.

### BACKGROUND

RNA interference (abbreviated as RNAi) refers to a phenomenon of gene silencing induced by double-stranded RNA in molecular biology, which is a mechanism of inhibiting gene expression by hindering the transcription or translation of specific genes. When a double-stranded RNA homologous to the coding region of an endogenous mRNA is introduced into cells, the mRNA will be degraded, leading to the silence of gene expression. Small interfering RNA (siRNA) with a length of 20-25 nt can trigger RNAi, which can specifically down-regulate or shut down the expression of specific genes, and has the characteristics of high efficiency, easy synthesis and easy operation. Therefore, this technology has been widely used in the field of exploration of gene function and gene therapy for infectious diseases and malignant tumors.

Hepatic fibrosis, as a result of liver injury, is the abnormal proliferation of connective tissue in the liver caused by various pathogenic factors. Hepatic fibrosis may lead to liver dysfunction and is the main process of liver injury developing into other chronic diseases. It is characterized by the recruitment of inflammatory cells and the activation of hepatic stellate cells (HSCs) in response to chronic injury, which results in the accumulation of extracellular matrix. Steatosis generally coexists with hepatitis and hepatocellular damage. Increased oxidative stress is a common cause of fibrosis in all chronic liver diseases, regardless of the cause of the disease. Damaged hepatocytes, HSCs and infiltrating inflammatory cells are the main sources of reactive oxygen species (ROS). In fact, oxidative stress will induce the recruitment of inflammatory cells and the activation of HSCs. Therefore, in the context of chronic liver injury, a vicious cycle of hepatocellular injury, production of ROS, activation of HSCs, and recruitment of inflammatory cells will occur, thereby amplifying the fibrotic response to injury.

In recent years, the development and treatment of anti-hepatic fibrosis drugs have made significant progress in terms of different targets of hepatic fibrosis, inhibiting the activation and proliferation of hepatic stellate cells, enhancing the activity of matrix metalloproteinases and inhibiting the activity of inhibitors of metalloproteinases tissue, inhibiting inflammation response, drugs for regulating the immune response, the combination therapy of nanoparticles with anti-fibrotic drugs and gene therapy. However, there is currently no effective treatment for hepatic fibrosis. Conventional drugs may have adverse reactions caused by factors such as large doses, while gene therapy has become an indispensable research direction for anti-hepatic fibrosis in the future due to its characteristics such as high efficiency, strong targeting, and easy operation.

Reactive oxygen species (ROS) can regulate the activation and expression of various mediators through redox-sensitive protein kinases and transcription factors in HSCs. NADPH oxidase is the main source of ROS in phagocytes and plays a key role. NOX is the catalytic subunit of NADPH oxidase, and NOX1 is a subtype of NOX that generates ROS. During the formation of hepatic fibrosis, various pathogenic factors continuously act on hepatic stellate cells (HSCs), causing their activation, proliferation, and reduced the apoptosis, thereby increasing the synthesis of collagen and the excessive accumulation of extracellular matrix (ECM). Therefore, if the activation and proliferation of HSCs are effectively inhibited, the occurrence and further development of hepatic fibrosis can be effectively prevented. Reactive oxygen species plays an important role in the formation of hepatic fibrosis, and the NADPH oxidase (NOX) functions mainly by activating HSC. Studies have shown that the expression of NOX1 is increased in rats with hepatic fibrosis and patients with hepatic cirrhosis. Studies further indicate that NOX1 may become a new therapeutic target for hepatic fibrosis (Lan, Kisseleva et al. 2015). Hepatic stellate cells (HSC) are located in the hepatic sinusoidal space, accounting for about 10% of the total number of liver cells. Generally, drugs can only enter the liver cells and rarely enter the hepatic sinusoidal space, and it is even more difficult to enter the hepatic stellate cells (HSCs). Therefore, there are few drugs targeting the hepatic stellate cells (HSCs).

### SUMMARY

The inventors of the present invention unexpectedly found that RNA interference (RNAi) by NOX1 siRNA can effectively target NOX1 protein and inhibit the production of reactive oxygen species (ROS), thereby playing a role in the treatment of hepatic fibrosis.

The technical solutions of the present invention involve a small interfering RNA that inhibits the expression of target gene NOX1, consisting of a sense strand and an antisense strand reversely complementary thereto, wherein sequences of the sense strand and the antisense strand are selected from the group consisting of:
(1) the sense strand has a nucleotide sequence of 5′-GAGAUGUGGGAUGAUCGUGACTT-3′(SEQ ID NO.1), and
the antisense strand has a nucleotide sequence of 5′-GUCACGAUCAUCCCACAUCUCTT-3′ (SEQ ID NO.2);
(2) the sense strand has a nucleotide sequence of 5′-CAAGCUGGUGGCCUAUAUGAUTT-3′ (SEQ ID NO.3), and

the antisense strand has a nucleotide sequence of 5′-AUCAUAUAGGCCACCAGCUUGTT-3′ (SEQ ID NO.4); and
(3) the sense strand has a nucleotide sequence of 5′-CUGAGUCUUGGAAGUGGAUCCUUTT-3′ (SEQ ID NO.5),
the antisense strand has a nucleotide sequence of 5′-AAGGAUCCACUUCCAAGACUCAGTT-3′ (SEQ ID NO.6);
wherein, the antisense strand is reversely complementary to a fragment of the target gene.

Further, in the small interfering RNA, the sense strand and the antisense strand are optionally modified with 2′-O-ribose modification on the first 21 nucleotides from the 5′ end, and the 2′-O-ribose modification is selected from the group consisting of 2′-O-ribose methylation modification, 2′-O-ribose fluoro modification and 2′-O-MOE modification.

Another object of the present invention is to provide an effective small interfering RNA delivery preparation.

The present invention provides the following technical solution:

A delivery preparation comprising the small interfering RNA of the present invention and a cationic polymer, wherein the small interfering RNA is carried by the cationic polymer and the cationic polymer is a polyethyleneimine polymer.

Further, in the delivery preparation of the present invention, the polyethyleneimine polymer has a molecular formula of

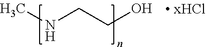

with a molecular weight of 40000-52000 Da, wherein n is determined according to the molecular weight.

Further, in the delivery preparation of the present invention, the polyethyleneimine polymer is in vivo-jet PEI® from Polyplus of France.

Further, the delivery preparation of the present invention comprises the small interfering RNA, the polyethyleneimine polymer and a solvent, wherein the small interfering RNA, the polyethyleneimine polymer and the solvent are in a ratio of 1 g:0.1-0.2 L:50-150 L.

Preferably, in the delivery preparation of the present invention, the small interfering RNA, the polyethyleneimine polymer and the solvent are in a ratio of 1 g:0.16 L:80-120 L.

Preferably, in the delivery preparation of the present invention, the solvent is 5% aqueous solution of glucose.

The present invention also provides therapeutic use of the small interfering RNA and the delivery preparation.

As a solution of the present invention, the present invention also provides use of the

small interfering RNA and the delivery preparation in the manufacture of a medicament for treating liver injury or hepatic fibrosis.

As another solution of the present invention, the present invention also provides use of the small interfering RNA and the delivery preparation in the manufacture of a medicament for reducing the expression level of NOX1 mRNA in the liver.

As another solution of the present invention, the present invention also provides use of the small interfering RNA and the delivery preparation in the manufacture of a medicament for treating or preventing acute cholestatic liver disease.

As another solution of the present invention, the present invention also provides use of the small interfering RNA and the delivery preparation in the manufacture of a medicament for treating or preventing pulmonary fibrosis.

As a solution of the present invention, the present invention also provides use of the small interfering RNA and the delivery preparation in the manufacture of a medicament for treating nonalcoholic fatty liver disease.

The present invention can achieve the following technical effects: in the context of no effective means for treating acute liver injury and hepatic fibrosis currently, the present invention has screened and isolated two small interfering RNA sequences through in-depth research, which can effectively reduce the expression level of NOX1 mRNA in the liver, and has been proved by experiments to show a therapeutic effect in carbon tetrachloride-induced acute liver injury and chronic hepatic fibrosis models and, additionally, an obvious improvement effect on α-naphthalene isothiocyanate (ANIT)-induced acute cholestatic liver disease in mice, bleomycin-induced pulmonary fibrosis in mice and high-fat diet-induced nonalcoholic fatty liver disease in mice. The present invention further utilizes the cationic polymer in vivo jet PEI® purchased from Polyplus company of France to carry the siRNA targeting NOX1, which achieves a good delivery effect. During the experiment, no obvious toxicity has been found.

### BRIEF DESCRIPTION OF DRAWINGS

FIG. 1 shows the effect of NOX1 siRNA 1 on ALT in CCl₄-induced acute liver injury model mice;

FIG. 2 shows the effect of NOX1 siRNA 1 on AST in CCl₄-induced acute liver injury model mice;

FIG. 3 shows the effect of NOX1 siRNA 1 on TBIL in CCl₄-induced acute liver injury model mice;

FIG. 4 shows the effect of NOX1 siRNA 1 on HE slices of liver of CCl₄-induced acute liver injury model mice;

FIG. 5 shows the effect of NOX1 siRNA 2 on ALT in CCl₄-induced chronic hepatic fibrosis model mice;

FIG. 6 shows the effect of NOX1 siRNA 2 on AST in CCl₄-induced chronic hepatic fibrosis model mice;

FIG. 7 shows the effect of NOX1 siRNA 2 on ALT/AST in CCl₄-induced chronic hepatic fibrosis model mice;

FIG. 8 shows the effect of NOX1 siRNA 2 on TBIL in CCl₄-induced chronic hepatic fibrosis model mice;

FIG. 9 shows the effect of NOX1 siRNA 2 on Sirius red slices of liver of CCl₄-induced chronic hepatic fibrosis model mice; and

FIG. 10 shows the effect of NOX1 siRNA 3 in improving bleomycin-induced pulmonary fibrosis in mice.

### DETAILED DESCRIPTION

The following examples are used to further illustrate the present invention in detail, but do not limit the present invention in any form.

In the present invention, CCl₄ was used to induce acute liver injury model and chronic hepatic fibrosis model in mice. CCl₄ is the most widely used chemical hepatotoxicant to induce hepatic fibrosis and hepatic cirrhosis in experimental animals. In the liver of model animals, CCl₄ is metabolized by cytochrome P450 into trichloromethyl free radical CCl₃• and chloride ion free radical Cl•. CCl₃• will covalently bind to macromolecules such as nucleic acid, protein and lipid in liver cells and triggers the generation of trichloromethyl peroxyl free radicals and the peroxidation of lipid, which will damage various membrane systems of liver cells (including endoplasmic reticulum, mitochondria and plasma membrane) and result in decreased permeability, thereby leading to the activation of the originally static hepatic stellate cells in the Disse space and further promoting the hepatic fibrosis.

In the present invention, the detection of serum biochemical indexes related to liver function of mice in the control group, the model group and the treatment group includes the detection of alanine aminotransferase (ALT), aspartate aminotransferase (AST) and total bilirubin (TBIL) in serum. When liver cells are treated with drugs or poisons that are toxic to the liver to cause damage or death, the permeability of the cell membrane increases, and the alanine aminotransferase (ALT) and aspartate aminotransferase (AST) in the cytoplasm enter the serum, resulting in the raised concentration in serum. Alanine aminotransferase (ALT) is mostly found in the liver, and aspartate aminotransferase (AST) is mainly found in the myocardium, followed by tissues such as liver, skeletal muscle and kidney. The significantly elevated aspartate aminotransferase and the ratio of aspartate aminotransferase/alanine aminotransferase (ALT) greater than 1 indicate extensive damage to the liver parenchyma. Total bilirubin (TBIL) is the sum of direct bilirubin and indirect bilirubin. Liver plays an important role in the metabolism of bilirubin, and the content of total bilirubin (TBIL) in serum can indirectly reflect the liver function.

### PREPARATION EXAMPLES 1-3

### Preparation of NOX1 siRNA 1-3

NOX1 siRNA with Sequence Nos. 1-3 were respectively synthesized with automated equipment by a solid-phase phosphoramidite chemical method including 4 cyclic steps, namely: deprotection, coupling, oxidation and capping.

This technology is a conventional technology in the prior art, which was carried out specifically as follows. The nucleoside at the 3′ end of the oligonucleotide chain of NOX1 siRNA with Sequence No. 1, 2 or 3 to be synthesized was first fixed on an insoluble macromolecule, and then the chain was gradually extended starting from this terminal nucleoside and constantly fixed on the carrier. Excess unreacted substances and decomposed products were removed by filtration or washing. Each extension went through a cycle. When the entire chain had extended to the required length, the oligonucleotide chain was cut off from the solid phase carrier and the protective group was removed. After separation and purification, the desired final product was obtained.

NOX1 siRNA with Sequence Nos. 1-3 were all prepared by a company of synthesis, and the sequences are as follows:
(1) Sequence No. 1:
the sense strand has a nucleotide sequence of 5′-GAGAUGUGGGAUGAUCGUGACTT-3′ (SEQ ID NO.1), and
the antisense strand has a nucleotide sequence of 5′-GUCACGAUCAUCCCACAUCUCTT-3′ (SEQ ID NO.2).
(2) Sequence No. 2:
the sense strand has a nucleotide sequence of 5′-CAAGCUGGUGGCCUAUAUGAUTT-3′ (SEQ ID NO.3), and
the antisense strand has a nucleotide sequence of 5′-AUCAUAUAGGCCACCAGCUUGTT-3′ (SEQ ID NO.4).
(3) Sequence No. 3:
the sense strand has a nucleotide sequence of 5′-CUGAGUCUUGGAAGUGGAUCCUUTT-3′ (SEQ ID NO.5),
the antisense strand has a nucleotide sequence of 5′-AAGGAUCCACUUCCAAGACUCAGTT-3′ (SEQ ID NO.6).

### EXAMPLE 1

### Effect of NOX1 siRNA with Sequence No. 1 on Carbon Tetrachloride-Induced Acute Liver Injury in Mice and Comparison with Positive Drug Silymarin

### Reagents and Instruments

CCl₄ (purchased from Shanghai Aladdin Biochemical Technology Co., Ltd., Cat No. C112040, batch No. B2027005), soybean oil (JinLongYu, Q/BBAH0019S), Nox1 siRNA with Sequence No. 1, silymarin (purchased from Dalian Meilun Biotechnology Co., Ltd., Cat No. MB1962, batch No. 00801A), CMC-Na (purchased from Beijing Solarbio Technology Co., Ltd., Cat No. C8620, batch No. 617O022), in vivo-jet PEI (Polyplus company of France), paraformaldehyde, liver tissue RNA extraction kit, and reverse transcription kit.

### Experimental Method

56 male C57BL/6 mice (Vital River), with a body weight of 24±1 g at the beginning of the experiment, were adaptively reared for one week under the conditions of a temperature of 24-26° C., a humidity of 60%, and light and dark for 12 hours each. CCl₄ and soybean oil were mixed at a volume ratio of 1:3. Each male C57BL/6 mouse except the control group was intraperitoneally injected with the solution of CCl₄ and soybean oil at 0.05 mL/10 g (body weight), and each mouse in the control group was injected with an equal volume of soybean oil. 24 hours after modeling, siRNA or silymarin were administered for the first time, and 48 hours later, the mice were sacrificed for sampling. The serum of the mice was collected, and the livers were frozen and fixed with 4% paraformaldehyde.

### Method of Administration

100 μl injection volume of a delivery preparation injection solution of 5% glucose was prepared according to the ratio of 0.16 μl in vivo-jet PEI per 1 μg RNA.
(1) 5 μg of RNA 1 was diluted in 25 μl of 10% glucose, added with 25 μl of ddH₂O, and vortexed gently.
(2) 0.8 μl of in vivo-jet PEI was diluted in 25 μl of 10% glucose, added with 25 μl of ddH₂O, and vortexed gently.
(3) The diluted in vivo-jet PEI was added to the diluted RNA and vortexed.
(4) Incubation was performed at room temperature for 15 min.

During the preparation process, the operator wore a mask, and the workbench was wiped with alcohol and RNA cleaner before use. Due to the loss during the administration process, an additional 100 μl of the drug should be prepared according to the number of mice to be administered.

### Grouping Design

TABLE 1
Instructions for grouping of experimental
animals and drug treatment

| Group | Number | Administration |
|---|---|---|
| Control | 8 | Soybean oil solution at 0.05 mL/ |
| group | | 10 g (weight) |
| Model | 12 | Solution of CCl₄ and soybean |
| group | | oil at 0.05 mL/10 g (weight) |
| Model | 12 | Solution of CCl₄ and soybean |
| group + | | oil at 0.05 mL/10 g (weight), |
| low-dose | | and No. 1 siRNA (5 μg/mouse) |
| siRNA | | by injection into the tail vein |
| | | after 24 h |
| Model | 12 | Solution of CCl₄ and soybean |
| group + | | oil at 0.05 mL/10 g (weight), |
| high-dose | | and No. 1 siRNA (15 μg/mouse) |
| siRNA | | by injection into the tail vein |
| | | after 24 h |
| Model | 12 | Solution of CCl₄ and soybean |
| group + | | oil at 0.05 mL/10 g (weight), |
| silymarin | | and silymarin (54 mg/kg/d) |
| | | by gavage after 24 h and 48 h |

Note:
Silymarin was prepared with 5‰ sodium carboxymethylcellulose.

### Detection Indicators

1. Body Weight and Observation of Survival Rate

Every day, the mice were weighed and the general conditions of the animals were observed and recorded, such as body weight, grasping stress, and behavioral activities of mouse.

2. Detection of Biochemical Indicators in Serum

The mice in all groups were sacrificed 48 hours after the administration, and the eyeballs were removed to collect the blood. The blood was centrifuged at 3000 r/min for 10 min to separate the serum, and then the levels of ALT, AST, and TBIL were determined with an automatic biochemical analyzer.

3. Histopathological Examination of Liver Tissue

48 hours after the administration, the mice were sacrificed, and the eyeballs were removed first to collect blood. After the mice were dead, they were dissected. The liver tissue was cut longitudinally, fixed with 10 times the volume of 4% paraformaldehyde, sliced at a thickness of 3-5 μm, embedded in paraffin, and stained with hematoxylin-eosin (HE). The pathological changes of the liver were observed under an optical microscope.

4. Detection of Molecular Biological Indicators

48 hours after the administration, the mice were sacrificed, and the eyeballs were removed first to collect blood. After the mice were dead, they were dissected, and the liver tissue of the mice was taken. RNA in the liver tissue was extracted, and reverse-transcribed into cDNA by reverse transcription PCR. The mRNA expression of NOX1, IL-1β, TNF-α and GAPDH in the liver tissue was determined by the real-time fluorescence quantitative PCR method. Primers:

NOX1-mouse
(Sense:
5′-CTG ACA AGT ACT ATT ACA CGA GAG-3′,
(SEQ ID NO. 7)
Antisense:
5′-CAT ATA TGC CAC CAG CTT ATG GAA G-3′
(SEQ ID NO. 8));
GAPDH-mouse
(Sense:
5′-TCA ACG GGA AAC CCA TCA CCA T-3′,
(SEQ ID NO. 9)
Antisense:
5′-GAA CAC GGA AGG CCA TGC CAG T-3′
(SEQ ID NO. 10));
IL-1β-mouse
(Sense: 5′-CCA TGG CAC ATT CTG TTC AAA-3′,
(SEQ ID NO. 11)
Antisense:
5′-GCC CAT CAG AGG CAA GGA-3′
(SEQ ID NO. 12));
TNF-α-mouse
(Sense: 5′-AGT CAA CCT CCT CTC TGC CG-3′,
(SEQ ID NO. 13)
Antisense:
5′-CTC CAA AGT AGA CCT GCC CG-3′
(SEQ ID NO. 14)).

5. Statistical Methods

Data analysis and statistical graphing were conducted using GraphPad Prism 8. Independent samples were tested by t test, where *P<0.05 indicated significant difference compared with the blank control group, **P<0.01 indicated extremely significant difference compared with the blank control group, #P<0.05 indicated significant difference compared with the model group, and ##P<0.01 indicated extremely significant difference compared with the model group, which had statistical significance.

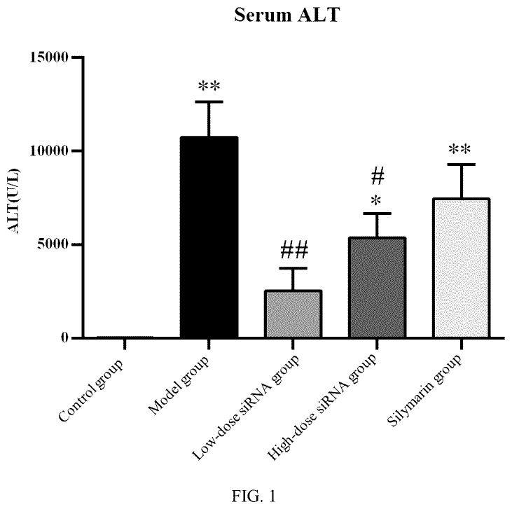

6. The results are shown in FIGS. 1-4.

As shown in FIG. 1, 48 hours after the intraperitoneal injection of CCl₄ in mice, the ALT level in the serum of mice in the model group was extremely significantly higher than that of the normal control group (**P<0.01); compared with the model group, the positive control drug silymarin group can reduce the ALT level in the serum, but there was no statistical difference; the low-dose NOX1 siRNA 1 group can significantly reduce the ALT level in the serum (##P<0.01), and the high-dose NOX1 siRNA 1 group can also reduce the ALT level (#P<0.05), showing a weaker effect than the low-dose NOX1 siRNA 1 group.

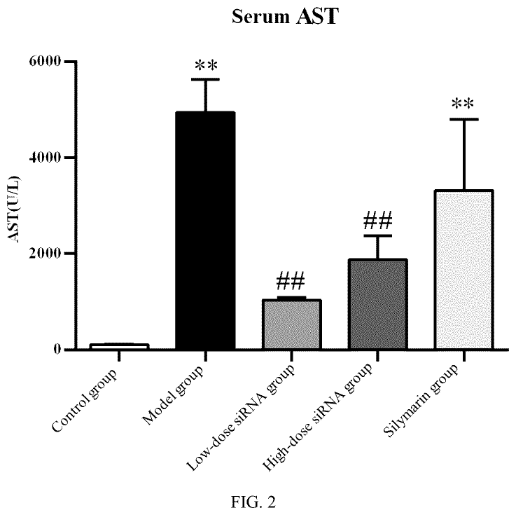

As shown in FIG. 2, 48 hours after the intraperitoneal injection of CCl₄ in mice, the AST level in the serum of mice in the model group was extremely significantly higher than that in the normal control group (**P<0.01); compared with the model group, the positive control drug silymarin group can reduce the AST level in the serum, but there was no statistical difference; both of the low-dose and high-dose No. 1 NOX1 siRNA groups can significantly reduce the AST level in the serum (##P<0.01), and the low-dose No. 1 NOX1 siRNA group had a stronger effect than the high-dose group.

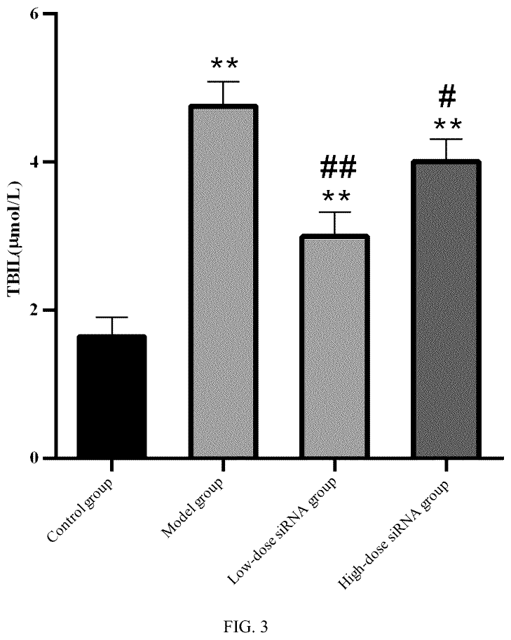

As shown in FIG. 3, 48 hours after the intraperitoneal injection of CCl₄ in mice, the TBIL level in the serum of mice in the model group was extremely significantly higher than that in the normal control group (**P<0.01); and both of the low-dose (##P<0.01) and high-dose (#P<0.05) NOX1 siRNA 1 groups can significantly reduce the TBIL level in the serum (##P<0.01).

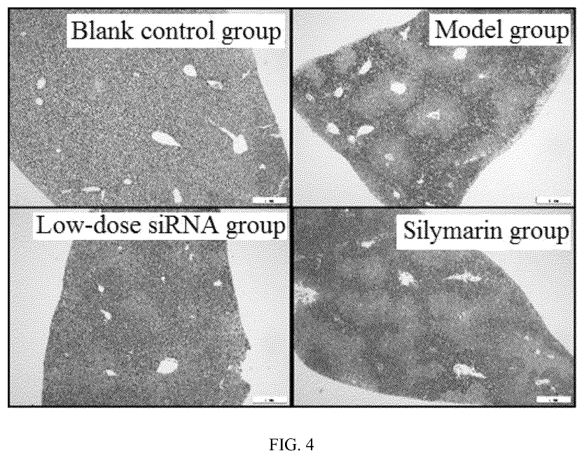

As shown in FIG. 4, 48 hours after the intraperitoneal injection of CCl₄ in mice, the results of the HE staining of liver tissue showed that compared with the control group, the model group had increased cell degeneration and necrosis; both of the low-dose Sequence No. 1 NOX1 siRNA group and the positive control drug silymarin group can reduce cell necrosis, and the low-dose Sequence No. 1 NOX1 siRNA group had a more obvious therapeutic effect.

### EXAMPLE 2

### Effect of NOX1 siRNA with Sequence Nos. 2 and 3 on Carbon Tetrachloride-Induced Acute Liver Injury in Mice and Comparison with Positive Drug Silymarin

The operation was the same as in Example 1 except that the RNA used was NOX1 siRNA with Sequence No. 2 or Nox1 siRNA with Sequence No. 3.

Test results: The obtained test data of the effect of NOX1 siRNA with Sequence No. 2 or NOX1 siRNA with Sequence No. 3 were comparable to those in Example 1.

### EXAMPLE 3

### Effect of NOX1 siRNA with Sequence No. 2 on Carbon Tetrachloride-Induced Chronic Hepatic Fibrosis Model in Mice and Comparison with Positive Drug Silymarin

### Reagents and Instruments

CCl₄ (purchased from Shanghai Aladdin Biochemical Technology Co., Ltd., Cat No. C112040, batch No. B2027005), soybean oil (JinLongYu, Q/BBAH0019S), NOX1 siRNA with Sequence No. 2, silymarin (purchased from Dalian Meilun Biotechnology Co., Ltd., Cat No. MB1962, batch No. 00801A), CMC-Na (purchased from Beijing Solarbio Technology Co., Ltd., Cat No. C8620, batch No. 6170022), in vivo-jet PEI, paraformaldehyde, liver tissue RNA extraction kit, reverse transcription kit, gavage needle, syringe, surgical instruments, inverted microscope, real-time fluorescence quantitative PCR instrument, PCR instrument, and serum biochemical analyzer.

### Experimental Method

68 male C57BL/6 mice (Vital River), with a body weight of 24±2 g at the beginning of the experiment, were adaptively reared for one week under the conditions of a temperature of 24-26° C., a humidity of 60%, and light and dark for 12 hours each. CCl₄ and soybean oil were mixed at a volume ratio of 2:5. Each male C57BL/6 mouse except the control group was intraperitoneally injected with the solution of CCl₄ and soybean oil at 0.02 mL/10 g (body weight) every three days, and each mouse in the control group was injected with an equal volume of soybean oil. The modeling was performed for 6 weeks. 2 weeks after the modeling, the test drugs were administered for 4 consecutive weeks (28 days), and the administration was stopped after 6 weeks.

### Method of Administration

The operation was the same as that in Example 1, except that the RNA was replaced with NOX1 siRNA with Sequence No. 2.

### Grouping Design

TABLE 2
Instructions for grouping of
experimental animals and drug treatment

| Group | Number | Administration |
|---|---|---|
| Control group | 8 | Soybean oil solution at 0.02 mL/10 g |
| | | (weight) |
| Model group | 12 | Solution of CCl₄ and soybean oil at |
| | | 0.02 mL/10 g (weight), and no |
| | | treatment after 2 weeks |
| Model group + | 12 | Solution of CCl₄ and soybean oil at |
| low-dose | | 0.02 mL/10 g (weight), and siRNA2 |
| siRNA with | | (2.5 μg/mouse) by injection into the |
| Sequence No. 2 | | tail vein every three days after 2 |
| | | weeks |
| Model group + | 12 | Solution of CCl₄ and soybean oil at |
| medium-dose | | 0.02 mL/10 g (weight), and siRNA2 |
| siRNA with | | (5 g/mouse) by injection into the tail |
| Sequence No. 2 | | vein every three days after 2 weeks |
| Model group + | 12 | Solution of CCl₄ and soybean oil at |
| high-dose | | 0.02 mL/10 g (weight), and siRNA2 |
| siRNA with | | (10 μg/mouse) by injection into the |
| Sequence No. 2 | | tail vein every three days after 2 |
| | | weeks |
| Model group + | 12 | Solution of CCl₄ and soybean oil at |
| silymarin | | 0.02 mL/10 g (weight), and silymarin |
| | | (54 mg/kg/d) by gavage every day |
| | | after 2 weeks |

Note:
Silymarin was prepared with 5‰ sodium carboxymethylcellulose.

### Detection Indicators

1. Body Weight and Observation of Survival Rate

Every three days, the mice were weighed and the general conditions of the animals were observed and recorded, such as body weight, grasping stress, and behavioral activities of mouse.

2. Detection of Biochemical Indicators in Serum

The mice in all groups were subjected to modeling constantly for 6 weeks and then sacrificed, and the eyeballs were removed to collect the blood. The blood was centrifuged at 3000 r/min for 10 min to separate the serum, and then the levels of ALT, AST, and TBIL were determined with an automatic biochemical analyzer.

3. Histopathological Examination of Liver Tissue

The mice were subjected to modeling constantly for 6 weeks and then sacrificed, and the eyeballs were removed first to collect blood. After the mice were dead, they were dissected. The liver tissue was cut longitudinally, fixed with 10 times the volume of 4% paraformaldehyde (need to be determined), sliced at a thickness of 3-5 μm, embedded in paraffin, and stained with hematoxylin-eosin (HE) and Sirius red. The pathological changes of the liver were observed under an optical microscope.

4. Detection of Molecular Biological Indicators

The mice were subjected to modeling constantly for 6 weeks and then sacrificed, and the eyeballs were removed first to collect blood. After the mice were dead, they were dissected. The liver tissue of the mice was taken. RNA in the liver tissue was extracted, and reverse-transcribed into cDNA by reverse transcription PCR. The mRNA expression of NOX1, IL-1β, TNF-α, α-SMA, Col-1α and GAPDH in the liver tissue was determined by the real-time fluorescence quantitative PCR method. Primers: NOX1-mouse (Sense: 5′-CTG ACA AGT ACT ATT ACA CGA GAG-3′ (SEQ ID NO.7), Antisense: 5′-CAT ATA TGC CAC CAG CTT ATG GAA G-3′ (SEQ ID NO.8)); GAPDH-mouse (Sense: 5′-TCA ACG GGA AAC CCA TCA CCA T-3′ (SEQ ID NO.9), Antisense: 5′-GAA CAC GGA AGG CCA TGC CAG T-3′ (SEQ ID NO.10)); IL-1β-mouse (Sense: 5′-CCA TGG CAC ATT CTG TTC AAA-3′ (SEQ ID NO.11), Antisense: 5′-GCC CAT CAG AGG CAA GGA-3′ (SEQ ID NO.12)); TNF-α-mouse (Sense: 5′-AGT CAA CCT CCT CTC TGC CG-3′ (SEQ ID NO.13), Antisense: 5′-CTC CAA AGT AGA CCT GCC CG-3′ (SEQ ID NO.14)); Col-1α-mouse (Sense: 5′-AAG GTT CTC CTG GTG AAG CTG GT-3′ (SEQ ID NO.15), Antisense: 5′-CTG AGC TCC AGC TTC TCC ATC TT-3′ (SEQ ID NO.16)); α-SMA (Sense: 5′-GAA GTA TCC GAT AGA ACA CGG CAT C-3′ (SEQ ID NO.17), Antisense: 5′-CCA GCA CAA TAC CAG TTG TAC GTC-3′ (SEQ ID NO.18)).

5. Statistical Methods

Data analysis and statistical graphing were conducted using GraphPad Prism 8. Independent samples were tested by t test, where *P<0.05 indicated significant difference compared with the blank control group, **P<0.01 indicated extremely significant difference compared with the blank control group, #P<0.05 indicated significant difference compared with the model group, and ##P<0.01 indicated extremely significant difference compared with the model group, which had statistical significance.

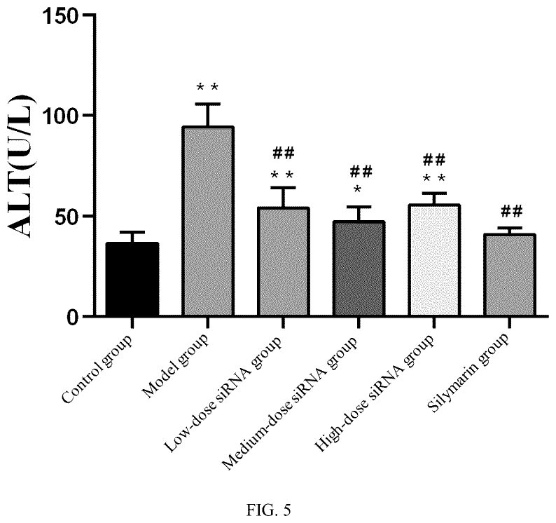

6. The results are shown in FIGS. 5-9.

As shown in FIG. 5, the mice were intraperitoneally injected with CCl₄ for 2 weeks of modeling followed by administration for 4 consecutive weeks. After a total of 6 weeks, the modeling and administration were finished. The ALT level in the serum of mice in the model group was extremely significantly higher than that of the normal control group (**P<0.01); compared with the model group, the positive control drug silymarin group can significantly reduce the ALT level in the serum (##P<0.01); all of the low-dose, medium-dose and high-dose NOX1 siRNA groups can significantly reduce the ALT level in the serum (##P<0.01), and the NOX1 siRNA medium-dose group had the most significant effect.

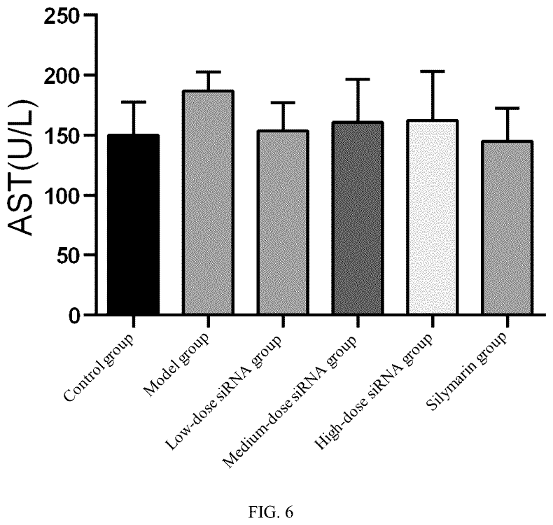

As shown in FIG. 6, the mice were intraperitoneally injected with CCl₄ for 2 weeks of modeling followed by administration for 4 consecutive weeks. After a total of 6 weeks, the modeling and administration were finished. The AST level in the serum of mice in the model group was extremely significantly higher than that in the normal control group (**P<0.01); compared with the model group, the positive control drug silymarin group can significantly reduce the AST level in the serum (##P<0.01); and all of the low-dose, medium-dose and high-dose NOX1 siRNA groups can also significantly reduce the AST level in the serum (##P<0.01) with similar effect.

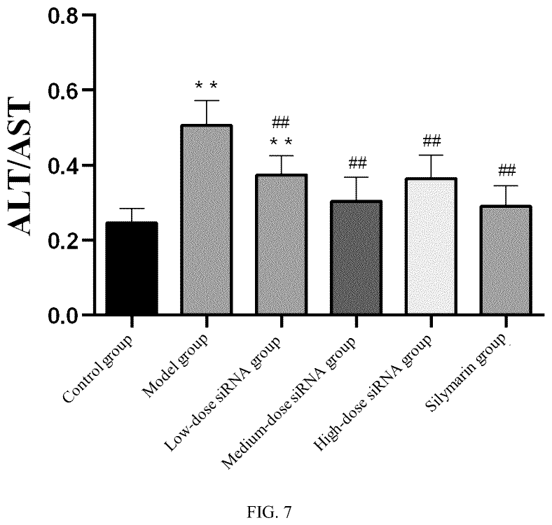

As shown in FIG. 7, the mice were intraperitoneally injected with CCl₄ for 2 weeks of modeling followed by administration for 4 consecutive weeks. After a total of 6 weeks, the modeling and administration were finished. The ALT/AST level in the serum of mice in the model group was extremely significantly higher than that in the normal control group (**P<0.01); compared with the model group, the positive control drug silymarin group can significantly reduce the ALT/AST level in the serum (##P<0.01); all of the low-dose, medium-dose and high-dose NOX1 siRNA groups can also significantly reduce the ALT/AST level in the serum (##P<0.01), and the NOX1 siRNA medium-dose group had the most significant effect.

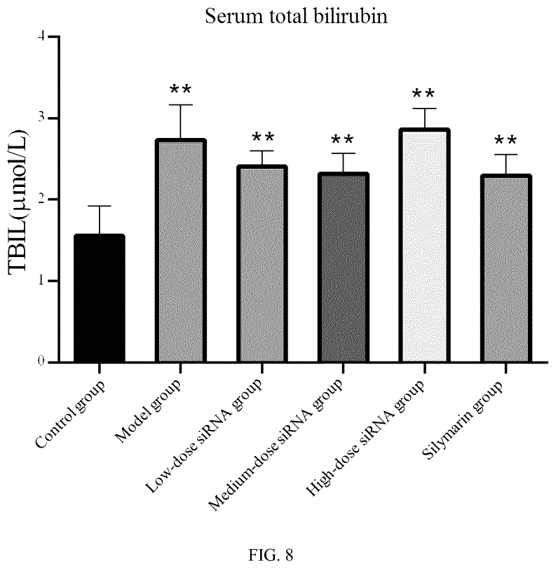

As shown in FIG. 8, the mice were intraperitoneally injected with CCl₄ for 2 weeks of modeling followed by administration for 4 consecutive weeks. After a total of 6 weeks, the modeling and administration were finished. The TBIL level in the serum of mice in the model group was extremely significantly higher than that in the normal control group (**P<0.01); compared with the model group, all of the positive control drug silymarin group and the low-dose and medium-dose NOX1 siRNA with Sequence No. 2 groups can reduce the TBIL level in the serum, but there was no statistical significance. The high-dose NOX1 siRNA with Sequence No. 2 group did not show obvious effect.

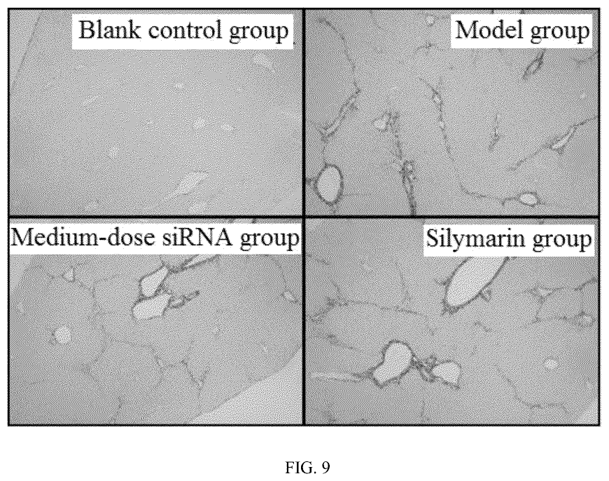

As shown in FIG. 9, the mice were intraperitoneally injected with CCl₄ for 2 weeks of

modeling followed by administration for 4 consecutive weeks. After a total of 6 weeks, the modeling and administration were finished. The results of the Sirius red staining of liver tissue showed that compared with the control group, the model group had obvious deposition and extension of type I collagen in the portal area, which was cross-linked with each other to form a pseudo-lobular structure. Both of the medium-dose NOX1 siRNA with Sequence No. 2 group and the positive control drug silymarin group can reduce the deposition of type I collagen in the portal area, and the medium-dose NOX1 siRNA with Sequence No. 2 group showed a more obvious effect.

### EXAMPLE 4

### Effect of NOX1 siRNA with Sequence No. 1 and No. 3 on Carbon Tetrachloride-Induced Chronic Hepatic Fibrosis Model in Mice and Comparison with Positive Drug Silymarin

The operation was the same as in Example 3 except that the RNA used was NOX1 siRNA with Sequence No. 1 or NOX1 siRNA with Sequence No. 3.

Test results: The obtained test data of the effect of NOX1 siRNA with Sequence No. 1 or NOX1 siRNA with Sequence No. 3 were comparable to those in Example 3.

### EXAMPLE 5

### Pharmacodynamic Effect of NOX1 siRNA with Sequence No. 3 on α-Naphthalene Isothiocyanate (ANIT)-Induced Acute Cholestatic Liver Disease Model in Mice

### Reagents and Instruments

ANIT (purchased from Sigma of USA, batch No. N4525), soybean oil (JinLongYu, Q/BBAH0019S), NOX1 siRNA 3, alanine aminotransferase (ALT) detection kit, aspartate aminotransferase (AST) detection kit, total bilirubin (TB) detection kit, direct bilirubin (DB) detection kit, total bile acid (TBA) detection kit and alkaline phosphatase (ALP) detection kit (Biosino Bio-technology Co., Ltd.).

### Experimental Method

In this experiment, normal male C57 mice were used, and 56 mice (Vital River) were divided into 4 groups according to body weight. The modeling and administration were carried out according to the table below:

TABLE 3
Instructions for grouping of experimental animals and drug treatment

| Group | Number | Administration | Modeling |
|---|---|---|---|
| Normal | 8 | / | |
| control | | | |
| group | | | |
| Model | 8 | Normal | ANIT was administered on the 4th |
| group | | saline (i.v.) | day, and the sample was collected |
| | | | after 48 hours, i.e., on the 6th day |
| Low-dose | 8 | i.v. | Both ANIT and drug were |
| siRNA | | (0.125 mg/kg) | administered on the 4th day, and the |
| group | | | sample was collected after 48 hours, |
| | | | i.e., on the 6th day |
| High-dose | 8 | i.v. | Both ANIT and drug were |
| siRNA | | (0.25 mg/kg) | administered on the 4th day, and the |
| group | | | sample was collected after 48 hours, |
| | | | i.e., on the 6th day |

The model group was administered with ANIT (dissolved in soybean oil) at 75 mg/kg on the 4th day. 48 hours after the administration of ANIT (i.e., on the 6th day, fasting for more than 6 hours), the animals were sacrificed. The low-dose and high-dose siRNA groups were administered with both drugs and ANIT (dissolved in soybean oil) at 75 mg/kg on the 4th day. 48 hours after administration of drugs and ANIT (i.e., on the 6th day, fasting for more than 6 hours), the animals were sacrificed.

After the experiment was completed, serum samples were collected to detect each

corresponding indicator to evaluate the pharmacodynamic efficacy. AST, ALT, total bilirubin (TB), direct bilirubin (DB), total bile acid (TBA) and ALP were determined through biochemical test of blood.

Data Analysis

After sorting and analyzing the experimental data, data analysis and table making were conducted using Office Excel 2019. Independent samples were tested by t test, where **P<0.01 indicated extremely significant difference compared with the blank control group, and ##P<0.01 indicated extremely significant difference compared with the model group, which had statistical significance.

The results are shown in Tables 4 and 5. A significant increase in ALT, AST, ALP, TBA, TB and DB in the serum of mice caused by ANIT indicated successful modeling. NOX1 siRNA 3 can reduce the levels of ALT, ALP and TBA in the serum at a high dose (0.25 mg/kg), which had significant statistical difference compared with the model group, indicating that NOX1 siRNA 3 had a certain protective effect on the cholestasis model induced by ANIT.

TABLE 4
Comparison of contents of ALT, AST, and ALP in the serum of mice (IU/L, x̅ ± s)

| Group | Number | ALT | AST | ALP |
|---|---|---|---|---|
| Control group | 10 | 50.90 ± 8.58 | 191.45 ± 19.89 | 159.94 ± 42.62 |
| Model group | 10 | 1747.40 ± 1167.77** | 5292.40 ± 2495.66** | 358.67 ± 85.47** |
| Low-dose siRNA | 10 | 1183.70 ± 346.16 | 4301.72 ± 1771.65 | 301.94 ± 59.85 |
| group | | | | |
| High-dose siRNA | 10 | 981.70 ± 430.92 # | 4130.54 ± 1861.44 | 204.04 ± 42.65## |
| group | | | | |

TABLE 5
Comparison of contents of TBA, TB and DB in the serum of mice (μmol/L, x̅ ± s)

| Group | Number | TBA | TB | DB |
|---|---|---|---|---|
| Control group | 10 | 12.18 ± 24.08 | 0.54 ± 0.29 | 1.19 ± 0.61 |
| Model group | 10 | 579.17 ± 265.13** | 124.82 ± 60.24** | 139.84 ± 66.04** |
| Low-dose siRNA group | 10 | 486.15 ± 127.35 | 96.55 ± 36.58 | 96.11 ± 41.61 |
| High-dose siRNA group | 10 | 345.28 ± 176.59# | 89.97 ± 33.21 | 93.30 ± 49.14 |

### EXAMPLE 6

### Effect of NOX1 siRNA with Sequence No. 3 in Improving Bleomycin-Induced Pulmonary Fibrosis in Mice

### Reagents and Instruments

Bleomycin (Zhejiang Hisun Pharmaceutical Co., Ltd., Chinese drug approval number: H20055883, 15 mg); hydroxyproline (HYP) kit (A030-2-1), and NOX1 siRNA with Sequence No. 3.

### Experimental Method

Grouping and modeling of animals: According to the random number table method, BALB/c mice were randomly divided into control group, model group and siRNA group, with 12 mice in each group. The mice were anesthetized by intraperitoneal injection of 1.5% pentobarbital sodium (2.5 mL/kg) and fixed on a table in a supine position. After cutting off the neck hair and ethanol sterilization, the trachea was exposed by separation layer by layer. A syringe was inserted into the trachea through the gap between the cartilage rings of the trachea towards the heart, and then 0.3 mL of bleomycin in normal saline solution (5 mg/kg) followed by 0.3 mL of air was injected. Immediately after the injection, the animal was kept upright and rotated left and right to evenly distribute the drug solution in the lung. The mice in the sham operation group were injected with an equal volume of normal saline in the lung. After 24 hours, the mice in the siRNA group were injected with siRNA with Sequence No. 3 (5 μg/mouse) into the tail vein, and the mice in the control group and the model group were injected with an equal volume of 10% aqueous solution of glucose. Thereafter, the mice were administered once a week. After 21 days, the mice were sacrificed to collect the lung tissues, which were quickly frozen in liquid nitrogen and stored in a −80° C. refrigerator for later use.

Statistical Methods

Data analysis and table making were conducted using Office Excel 2019. Independent samples were tested by t test, where **P<0.01 indicated extremely significant difference compared with the blank control group, and ##P<0.01 indicated extremely significant difference compared with the model group, which had statistical significance.

The results are shown in Table 6. NOX1 siRNA No. 3 can significantly reduce the

increase in hydroxyproline in the bleomycin-induced lung tissue, suggesting that it had an anti-pulmonary fibrosis effect.

TABLE 6
Comparison of content of hydroxyproline in lung
tissue of mice in each group on Day 21 (μg/mg, x̅ ± s)

| Group | Number | HYP |
|---|---|---|
| Control group | 12 | 0.13 ± 0.07 |
| Model group | 12 | 0.31 ± 0.11** |
| siRNA group | 12 | 0.17 ± 0.09## |

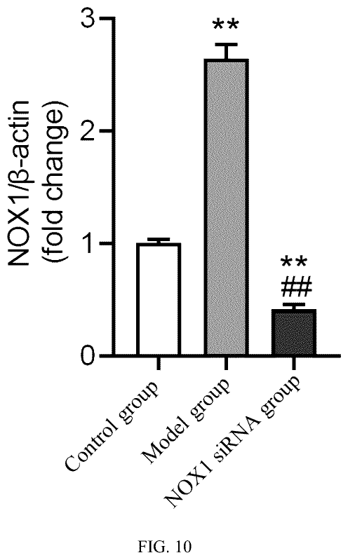

As shown in FIG. 10, bleomycin can induce the expression of NOX1 gene in lung tissue, and NOX1 siRNA with Sequence No. 3 can significantly reverse this process.

### EXAMPLE 7

### Effect of NOX1 siRNA with Sequence No. 3 in Improving High-Fat Diet-Induced Non-Alcoholic Fatty Liver Disease in Mice

### Reagents and Instruments

High-fat feed (Research Diets, model: D12492, containing 60% milk fat), fructose, sucrose, NOX1 siRNA with Sequence No. 3, microplate reader, analytical balance, syringes, and surgical instruments.

Sugar (fructose:sucrose=55%:45%) was added at 42 g/L to the drinking water of the mice in the model group, for example, 1 L of water should be added with 23.1 g fructose+18.9 g sucrose. The mice in the control group were provided with normal water.

### Preparation of RNA Drug

The drug injection solution was prepared according to the body weight of the mice at 50 μl/10 g, then provided to Qingda Kerui Biotechnology Co., Ltd once a week (the drug was prepared according to 30 g of mouse body weight to ensure a sufficient amount), and stored at 4° C.

Preparation of 100 μl of injection volume of 5% glucose containing 1 μg of RNA (adjusted according to the actual drug amount administered to the mice) and 0.24 μl of in vivo-jet PEI (adjusted according to the actual drug amount administered to the mice):
(1) 1 μg of RNA was diluted into 25 μl of 10% glucose, added with 25 μl of DEPC water, and vortexed gently.
(2) 0.24 μl of in vivo-jet PEI was diluted into 25 μl of 10% glucose, added with 25 μl of DEPC water, and vortexed gently.
(3) The diluted in vivo-jet PEI was added into the diluted RNA and vortexed.
(4) Incubation was performed at room temperature for 15 min.

### Experimental Method

50 male C57BL/6J mice (SPF grade) aged 8-10 weeks with a body weight of 20±1 g were reared under the conditions of a temperature of 24-26° C., a humidity of 60%, and light and dark for 12 hours each.

After one week of adaptive rearing, a total of 10 mice in the control group were fed with normal feed. 40 male C57BL/6J mice were subjected to modeling by feeding HFHF, and during this period, the mice had free access to food and drinks. The modeling proceeded constantly for 16 weeks. During this period, the survival of the mice was observed every day, and the body weight was recorded once a week. After 12 weeks, the mice were administered with test drug for 4 consecutive weeks, and the administration was stopped after 16 weeks.

### Grouping Design and Drug Treatment

TABLE 7
Instructions for grouping of experimental animals and drug treatment

| Group | Number | Administration |
|---|---|---|
| Control group | 10 | Normal feed, and normal saline by injection |
| | | into the tail vein every three days after 12 |
| | | weeks |
| Model group | 10 | HFHF, and normal saline by injection into |
| | | the tail vein every three days after 12 weeks |
| Model group + | 10 | HFHF, and siRNA (0.125 μg/g) by injection |
| low-dose | | into the tail vein every three days after 12 |
| siRNA | | weeks |
| Model group + | 10 | HFHF, and siRNA (0.25 μg/g) by injection |
| medium-dose | | into the tail vein every three days after 12 |
| siRNA | | weeks |
| Model group + | 10 | HFHF, and siRNA (0.5 μg/g) by injection |
| high-dose | | into the tail vein every three days after 12 |
| siRNA | | weeks |

Statistical Methods

Data analysis and table making were conducted using Office Excel 2019. Independent samples were tested by t test, where **P<0.01 indicated extremely significant difference compared with the blank control group, and ##P<0.01 indicated extremely significant difference compared with the model group, which had statistical significance.

Table 8 shows that a high-fat and high-sugar diet can significantly increase the content of TC and TG in the liver tissue of the mice, suggesting lipid deposition in the liver tissue. NOX1 siRNA can reduce the content of TC in liver tissue, and the high-dose group had a significant difference compared with the model group (P<0.05). All of the low-, medium- and high-dose NOX1 siRNA No. 3 groups can reduce the content of TG in liver tissue.

Table 9 shows that a high-fat and high-sugar diet can significantly increase ALT and AST in the serum of the mice, indicating liver cell injury. The elevated levels of TC and TG in the serum indicated that the capacity of metabolizing lipid of the liver was overloaded, indicating successful modeling. NOX1 siRNA can reduce the content of TC in the serum, and the medium- and high-dose groups had significant difference compared with the model group (#P<0.05, ##P<0.01). The high-dose NOX1 siRNA with Sequence No. 3 group can reduce the content of TG in the serum. The effect of NOX1 siRNA on the content of TC in the serum was not statistically different.

TABLE 8
Comparison of contents of TC and TG in
liver tissue of mice (mmol/g protein, x̅ ± s)

| Group | Number | TC | TG |
|---|---|---|---|
| Control group | 10 | 1.37 ± 0.32 | 1.13 ± 0.25 |
| Model group | 10 | 2.28 ± 0.40** | 1.81 ± 0.32** |
| Low-dose | 10 | 2.17 ± 0.69 | 0.95 ± 0.33## |
| siRNA group | | | |
| Medium-dose | 10 | 1.98 ± 0.51 | 1.17 ± 0.40## |
| siRNA group | | | |
| High-dose | 10 | 1.58 ± 0.30## | 0.92 ± 0.25## |
| siRNA group | | | |

TABLE 9
Comparison of contents of ALT, AST, TC and TG in serum of mice (x̅ ± s)

| Group | Number | ALT (IU/L) | AST (IU/L) | TC (mmol/L) | TG (mmol/L) |
|---|---|---|---|---|---|
| Control group | 10 | 53.90 ± 13.32 | 122.6 ± 28.39 | 1.90 ± 0.38 | 0.39 ± 0.06 |
| Model group | 10 | 163.0 ± 52.34** | 259.7 ± 76.38** | 5.48 ± 2.30** | 0.63 ± 0.18* |
| Low-dose siRNA | 10 | 168.5 ± 76.31 | 280.9 ± 65.33 | 5.34 ± 1.75 | 0.54 ± 0.29 |
| group | | | | | |
| Medium-dose | 10 | 107.4 ± 36.07# | 264.7 ± 43.51 | 4.72 ± 1.68 | 0.46 ± 0.19 |
| siRNA group | | | | | |
| High-dose siRNA | 10 | 107.5 ± 37.00# | 209.7 ± 66.67# | 4.24 ± 1.71 | 0.33 ± 0.11## |
| group | | | | | |

1. A small interfering RNA that inhibits the expression of target gene NOX1, consisting of a sense strand and an antisense strand reversely complementary thereto, wherein sequences of the sense strand and the antisense strand are selected from the group consisting of:
(1) sequences of No. 1 small interfering RNA
the sense strand has a nucleotide sequence of 5′-GAGAUGUGGGAUGAUCGUGACTT-3′ (SEQ ID NO.1), and
the antisense strand has a nucleotide sequence of 5′-GUCACGAUCAUCCCACAUCUCTT-3′ (SEQ ID NO.2);
(2) sequences of No. 2 small interfering RNA
the sense strand has a nucleotide sequence of 5′-CAAGCUGGUGGCCUAUAUGAUTT-3′ (SEQ ID NO.3), and
the antisense strand has a nucleotide sequence of 5′-AUCAUAUAGGCCACCAGCUUGTT-3′ (SEQ ID NO.4); and
(3) sequences of No. 3 small interfering RNA
the sense strand has a nucleotide sequence of 5′-CUGAGUCUUGGAAGUGGAUCCUUTT-3′ (SEQ ID NO.5),
the antisense strand has a nucleotide sequence of 5′-AAGGAUCCACUUCCAAGACUCAGTT-3′ (SEQ ID NO.6);
wherein, the antisense strand is reversely complementary to a fragment of the target gene.

2. The small interfering RNA according to claim 1, wherein the sense strand and the antisense strand are optionally modified with 2′-O-ribose modification on the first 21 nucleotides from the 5′ end, and the 2′-O-ribose modification is selected from the group consisting of 2′-O-ribose methylation modification, 2′-O-ribose fluoro modification and 2′-O-MOE modification.

3. A delivery preparation comprising the small interfering RNA according to claim 1 and a cationic polymer, wherein the small interfering RNA is carried by the cationic polymer and the cationic polymer is a polyethyleneimine polymer.

4. The delivery preparation according to claim 3, wherein the polyethyleneimine polymer has a molecular formula of 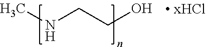
with a molecular weight of 40000-52000 Da, wherein n is determined according to the molecular weight.

5. The delivery preparation according to claim 4, wherein the polyethyleneimine polymer is in vivo-jet PEI® from Polyplus of France.

6. The delivery preparation according to claim 3, comprising the small interfering RNA, the polyethyleneimine polymer and a solvent, wherein the small interfering RNA, the polyethyleneimine polymer and the solvent are in a ratio of 1 g:0.1-0.2 L:50-150 L.

7. The delivery preparation according to claim 6, wherein the solvent is 5% aqueous solution of glucose.

8. A method for treating liver injury or hepatic fibrosis, comprising administering to a subject in need thereof the small interfering RNA according to claim 1.

9. A method for treating nonalcoholic fatty liver disease, comprising administering to a subject in need thereof the small interfering RNA according to claim 1.

10. A method for treating or preventing pulmonary fibrosis, comprising administering to a subject in need thereof the small interfering RNA according to claim 1.

11. A method for treating or preventing acute cholestatic liver disease, comprising administering to a subject in need thereof the small interfering RNA according to claim 1.


SEQUENCE LISTING (18 sequences)

SEQ ID NO: 1 - DNA, 23 bp, Artificial Sequence
  sense strand
gagaugugggaugaucgugactt

SEQ ID NO: 2 - DNA, 23 bp, Artificial Sequence
  antisense strand
gucacgaucaucccacaucuctt

SEQ ID NO: 3 - DNA, 23 bp, Artificial Sequence
  sense strand
caagcugguggccuauaugautt

SEQ ID NO: 4 - DNA, 23 bp, Artificial Sequence
  antisense strand
aucauauaggccaccagcuugtt

SEQ ID NO: 5 - DNA, 25 bp, Artificial Sequence
  sense strand
cugagucuuggaaguggauccuutt

SEQ ID NO: 6 - DNA, 25 bp, Artificial Sequence
  antisense strand
aaggauccacuuccaagacucagtt

SEQ ID NO: 7 - DNA, 24 bp, Artificial Sequence
  sense strand
ctgacaagtactattacacgagag

SEQ ID NO: 8 - DNA, 25 bp, Artificial Sequence
  antisense strand
catatatgccaccagcttatggaag

SEQ ID NO: 9 - DNA, 22 bp, Artificial Sequence
  sense strand
tcaacgggaaacccatcaccat

SEQ ID NO: 10 - DNA, 22 bp, Artificial Sequence
  antisense strand
gaacacggaaggccatgccagt

SEQ ID NO: 11 - DNA, 21 bp, Artificial Sequence
  sense strand
ccatggcacattctgttcaaa

SEQ ID NO: 12 - DNA, 18 bp, Artificial Sequence
  antisense strand
gcccatcagaggcaagga

SEQ ID NO: 13 - DNA, 20 bp, Artificial Sequence
  sense strand
agtcaacctcctctctgccg

SEQ ID NO: 14 - DNA, 20 bp, Artificial Sequence
  antisense strand
ctccaaagtagacctgcccg

SEQ ID NO: 15 - DNA, 23 bp, Artificial Sequence
  sense strand
aaggttctcctggtgaagctggt

SEQ ID NO: 16 - DNA, 23 bp, Artificial Sequence
  antisense strand
ctgagctccagcttctccatctt

SEQ ID NO: 17 - DNA, 25 bp, Artificial Sequence
  sense strand
gaagtatccgatagaacacggcatc

SEQ ID NO: 18 - DNA, 24 bp, Artificial Sequence
  antisense strand
ccagcacaataccagttgtacgtc